(19) United States
(12) Patent Application Publication
(10) Pub. No.: US 2009/0198173 A1
(43) Pub. Date: Aug. 6, 2009

(54) Light Emitting Device for use in Therapeutic and/or Cosmetic Treatment

(75) Inventors: Ifor David William Samuel, Fife (GB); James Ferguson, Fife (GB); Andrew Paul McNeill, Fife (GB)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Lumicure Limited, Fife (GB)

(21) Appl. No.: 12/298,496

(22) PCT Filed: Apr. 26, 2007
(86) PCT No.: PCT/GB2007/001549

§ 371 Date: Feb. 17, 2009

(30) Foreign Application Priority Data
Apr. 27, 2006 (GB) 0608315.8

(51) Int. Cl. A61N 5/06

(52) U.S. Cl. 604/20; 607/88; 607/93

(57) ABSTRACT

An ambulatory device for use in therapeutic and/or cosmetic treatment has a localised light source such as an LED (e.g. 6; 19; 46; 119; 219). Light from the source is output through an output surface which, in use, covers the area to be treated, and light is distributed over that surface by means of a diffusing member (e.g. 14; 114; 214) of which the output surface may form an integral part.

### FIELD OF THE INVENTION

This invention relates to a device for use in therapeutic and/or cosmetic treatment, particularly a treatment that involves exposure of part of the body to electromagnetic radiation. The invention also relates to such a device and a photo therapeutic agent for use therewith.

### BACKGROUND TO THE INVENTION

Light can be used to treat a wide variety of diseases. When light alone is used to treat a disease, the treatment is referred to as phototherapy. Light may be used in conjunction with a pharmaceutical in which case the treatment is called photodynamic therapy (PDT).

These therapies can be used to treat a variety of skin and internal diseases. In PDT, a light-sensitive therapeutic agent known as a photopharmaceutical is supplied externally or internally to an area of the body, which is to be treated. That area is then exposed to light of a suitable frequency and intensity to activate the photopharmaceutical. A variety of photopharmaceutical agents are currently available.

For example there are topical agents such as 5-aminolevulinic acid hydrochloride (Crawford Pharmaceuticals), methylaminolevulinic acid (Metfix), Photocure and Galderma. There are also injectable drugs used primarily for internal malignancies, including Photofrin (from Axcan) and Foscan (from Biolitech Lid). Often, the drug is applied in a non-active form that is metabolised to a light-sensitive photopharmaceutical.

In photodynamic therapy, the primary technique for supplying light to the photopharmaceutical is to project light of a suitable wavelength from standalone light sources such as lasers or filtered arc lamps, where the lamps are positioned some distance from the area to be treated. These sources are cumbersome and expensive, and are therefore only suitable for use in hospitals. This leads to inconvenience for the patient, and high cost for the treatment. High light irradiances are needed in order to treat an acceptable number of patients per day (for the treatment to be cost effective) and to avoid unduly inconveniencing the patient. PDT ideally requires that the area to be treated is uniformly illuminated which can be a problem with large area light sources placed at some distance from the patient.

Light emitting diodes (LEDs) are potentially an alternative, because they are lightweight and relatively cheap, and can therefore be used in ambulatory devices. However, they are intrinsically point sources whereas an area illuminator is required.

Attempts to solve this problem have involved arrays of large numbers of LEDs. Such arrangements are cumbersome and intrinsically use large amounts of LEDs.

WO 98/46130 and U.S. Pat. No. 6,096,066 (Chen and Wiscombe) disclose arrays of LEDs for use in photodynamic therapy. These arrays contain large numbers of LEDs for direct illumination of the area to be treated. The large number of devices consequently requires a suitably large power supply and can collectively generate a considerable amount of heat.

GB 2360461 (Whitehurst) discloses a flexible garment that uses a conventional photodynamic therapy light source to produce light that is then transmitted through optical fibres.

U.S. Pat. No. 5,698,866 (Doiron et al) disclose a light source using arrays of over-driven inorganic LEDs to directly illuminate the area to be treated. The device requires large numbers of LEDs and the resulting light output is not even. Because of the large number of devices a suitable mains powered electrical supply is required and consequently a heat-sinking mechanism, the device is suitable only for hospital treatment.

WO 93/21842 (Bower et al) discloses light sources using inorganic LEDs. The device uses large numbers of LEDs in an array to directly illuminate the area to be treated. Although transportable, the device is not suitable for ambulatory use by a patient at home and clinical treatment is envisaged. A further problem with existing approaches is that it can be difficult to achieve uniform illumination with such sources, especially on curved body parts.

WO 93/21842 (Bower et al) discloses inorganic LED arrays for direct illumination of the area to be treated. The number of LEDs are so great that the power requirements of such a device are specifically described as requiring mains power.

U.S. Pat. No. 5,616,140 (Prescott et al) disclose a battery operated, portable laser bandage having one or many lasers applied to a specific treatment area. These lasers are directed directly towards the area to be treated, the only solution to the problem of increasing the area of coverage being to provide more lasers.

US2005 070976 (Samuel and Ferguson) discloses using large area organic LEDs for the illumination of the area to be treated so that the entire surface of the device emits light. However, it would be desirable to achieve the illumination of a large area using other types of sources, of a more localised nature.

### SUMMARY OF THE INVENTION

According to the invention, there is provided an ambulatory device for use in therapeutic and/or cosmetic treatment, the device comprising a localised light source and a diffusing member for distributing light from the source over an area to be treated so as to illuminate, and cause said treatment of, that area.

The diffusing member increases the area that can be illuminated by a given source so that the number of sources required to perform an effective treatment of a given area can be reduced, thereby also reducing the power requirements of, and/or heat generated by, such a device.

The light source may be point-like (such as an inorganic LED) or may emit light over a larger area (for example, as would be the case with a fluorescent tube). In either case the source is localised in that it emits light over an area smaller than that to be treated, and the term ‘localised’ should therefore be construed accordingly.

Preferably, the diffusing member has an output surface which, in use, covers the area to be treated, said surface defining an emitting area across which light from the source is emitted by the device.

In at least one embodiment of the device, the diffusing member is made from a flexible material so that it is capable of conforming to the area to be treated.

Preferably, the source is situated behind said output surface so that substantially all the light emitted by the device passes through at least part of the diffusing member, the area of the output surface being greater than that of the source.

Since all the light to be emitted has passed through the diffusing member, the latter helps to avoid unacceptable variations in the intensity of light illuminating an area to be treated.

Preferably, the output surface has an area of at least one square centimetre, the said area preferably being in the range of 3-400 cm².

The light source may be spaced from the diffusing member so as to shine light directly or indirectly on to the latter. Alternatively, the light source is at least partially accommodated in a recess in the diffusing member, in which case the light source is preferably embedded within the diffusing member.

It has been found that this arrangement leads to a compact construction of device which distributes light across the output surface very effectively. In addition, the diffusing member can help to provide structural support and/or protection for the light source.

The device may have a single light source, preferably symmetrically situated relative to the output surface.

Alternatively, the light source may be one of a plurality of such light sources, preferably arranged in an array which is symmetrical relative to the output surface.

Where the device has a plurality of sources, each source may to advantage be at least partially accommodated in a respective recess in the diffusing member, and is more preferably embedded in the latter.

The diffusing member preferably has any number of sources between 1 and 30 inclusive. More preferably the possible number of sources lies in the range of 1 to 12 inclusive.

Preferably, the source or at least some of the sources are situated at or around the periphery of the diffusing member. For example, where there is a plurality of sources, one or more sources could be situated in the region of the centre of the member, the remaining sources being at the periphery, or all of the sources may be at the periphery of the diffusing member.

The or each source may to advantage comprise a light emitting semi-conductor device, preferably an LED.

The diffusing member may distribute light in any suitable way. For example the diffusing member may be such as to distribute light by scattering light in all directions from the source, may be such as to conduct or reflect light to discrete zones on the member from which the light is emitted, or may combine these two approaches. Thus the diffusing member may comprise a substantially homogenous body of translucent material all of which will scatter the light, or may have one or more light guides for supplying light to emission zones (which may include formations for scattering light) distributed across the member.

The diffusing member may comprise a sheet of diffusing material, preferably having a flat face. The flat face helps to achieve even distribution of light across the output area. The term ‘flat’ includes, for the purposes of this case, a surface which on a length scale of 1 mm appears flat discounting small features (for example, corrugations) of a size less than 1 mm in height.

The thickness of the sheet preferably decreases with increasing distance from the source or sources so as to compensate for the inverse relationship between the intensity of the light emitted from the source or sources with distance therefrom.

This variation in thickness also contributes to sufficiently even illumination being achieved.

Preferably, said decrease in thickness is progressive.

In such case, the device may have a plurality of sources arranged around the periphery of the diffusing member, the latter having a concave surface.

Alternatively, the diffusing member may comprise a rod, in which case the light source is preferably situated at one end of the rod.

The rod may to advantage be flexible.

Preferably, the device is for use in the treatment of a human or animal patient by photodynamic therapy. Preferably, the light generating semiconductor device emits light in the wavelength range of 300-900 nm and typically having a wavelength of 650 nm. The device may have LEDs of different wavelengths. These may be illuminated simultaneously or at separate times. The effective distribution of light from said light generating semiconductor device(s) can enable the number of light generating semiconductor device(s) required to be kept to a minimum, thereby reducing the weight of the device and the electrical power requirements, meaning the device can readily be powered by portable low voltage power supplies, such as batteries, forming a totally self-contained portable unit. The heat generated by the device is also reduced compared with devices having more light sources illuminating the same size of area. Indeed, the therapeutic device may to advantage include a power supply for operating the light-emitting semiconductor. The device is sufficiently portable to enable ambulatory treatment i.e. treatment in which the patient can move around freely. It can be subsequently removed in the patient's own time, so that treatment could take place at home or at work. This gives greater convenience and lower cost (from avoiding either an out-patient or in-patient stay in hospital). It also means that lower light levels can be used since exposure can occur for a longer period of time. This overcomes a problem of pain induced in some patients by the high irradiances from conventional sources used in hospitals. In addition lower irradiance is more effective in PDT due to reduction of the extent of photobleaching of the photopharmaceutical.

In at least one embodiment of the device the diffusing material is thinner at a point on the light-emitting area that is furthest from the light generating semiconductor device(s) light sources. This thinning of the diffusing material means that light can be emitted from the light-emitting area in a more even manner.

Preferably, the diffusing material distributes the light from light generating semiconductor device point sources across the emitting area of the device, providing continuous light emission. An output surface as large as 400 cm² might be square, e.g. 1 cm×1 cm, 2 cm×2 cm, 5 cm×5 cm, 10 cm×10 cm, or circular.

The device may be planar, or may be curved in advance or in situ to conform to the surface of the area to be exposed to light from the light-emitting semiconductor.

Preferably, the device is flexible so as to be capable of being formed into any of a number of possible different configurations in advance or extemporaneously to the shape of the treatment area to which it is to be applied. The device may be disposable, i.e. used to deliver one treatment and then thrown away.

The device may be used as a light emitting rod or cylinder, for example a diffusing rod of (but not limited to) 1.25-2.25 cm radius of say (but not limited to) 10-12 cm length for use inside the esophagus or other internal body structure.

Where the diffusing rod is flexible it may be formed into any of a number of possible different configurations in advance or extemporaneously to the shape of the treatment area to which it is to be applied.

The device conveniently includes an adhesive surface for attaching the device to a patient.

The devices may be provided with a photochemical and/or a photopharmaceutical preparation present. This may be in the form of a gel, ointment or cream. Alternatively, or as well, the device may be provided with a thin film impregnated with the photopharmaceutical. Typically, the photopharmaceutical preparation is provided as a layer in contact with the light source. Provided that the photopharmaceutical preparation is transparent or sufficiently translucent for the frequency of stimulating light the resulting device can be readily applied without a separate step of applying the photopharmaceutical to a patient. Creams that would scatter the light may nevertheless be used if they are absorbed before the light source is switched on. A photopharmaceutical layer may be covered by a peelable release medium, such as a silicone-backed sheet. The photopharmaceutical preparation may comprise an inactive compound that is metabolised in vivo to an active compound. Delivery of the photopharmaceutical can be assisted by iontophoresis. The output of light from the light-emitting semiconductor may be pulsed and an electronic control circuit or microprocessor may be provided to control this pulsing and/or other aspects of device function such as duration of exposure(s) of the area to be treated and the intensity of emitted light. Pulsed devices may be provided with a preparation of a photochemical and/or photopharmaceutical substance which is photobleachable or which is metabolised in vivo to a photobleachable chemical species.

An alternative type of diffusing member comprises a body of the patterned diffusing material described in WO2005101070 and which thus includes light guides for conveying light from the source(s) to emission zones along the guides.

### BRIEF DESCRIPTION OF THE DRAWINGS

The invention will now be described, by way of example only, with reference to the accompanying drawings in which:—

FIG. 1 is a cut away side view of a first embodiment of ambulatory device in accordance with the invention;

FIG. 2 is a plan view (not to scale) of the embodiment shown in FIG. 1.

FIG. 3 is a partially cut away plan view of a second embodiment of ambulatory device in accordance with the invention;

FIG. 4 is a cut away side view of the embodiment shown in FIG. 3;

FIGS. 5 and 6 show alternative ways of connecting that embodiment to a power source and controller;

FIGS. 7 and 8 are views, respectively corresponding to FIGS. 3 and 4, of a third embodiment of ambulatory device in accordance with the invention;

FIG. 9 is a cut away detailed view of part of the second embodiment;

FIG. 10 is a view corresponding to FIG. 1, of a fourth embodiment of ambulatory device in accordance with the invention;

FIG. 11 is a plan view of a fifth embodiment of ambulatory device in accordance with the invention;

FIG. 12 is a cut away side view of a sixth embodiment of ambulatory device in accordance with the invention, the device being for illumination of internal cavities of the body such as the esophagus and colon;

FIG. 13 is a cut away plan view of a seventh embodiment of ambulatory device in accordance with the invention;

FIG. 14 shows, by way of example, a selection of different types of LED which can be used in a device according to the invention.

FIG. 15 shows, in cut away side view, a modification to the embodiment shown in FIG. 10;

FIG. 16 shows a modification to the embodiment shown in FIG. 13.

FIG. 17 is a cut away side view showing a device, in accordance with the invention, having adhesive attachment means for attaching the device to the skin of a patient.

### DETAILED DESCRIPTION

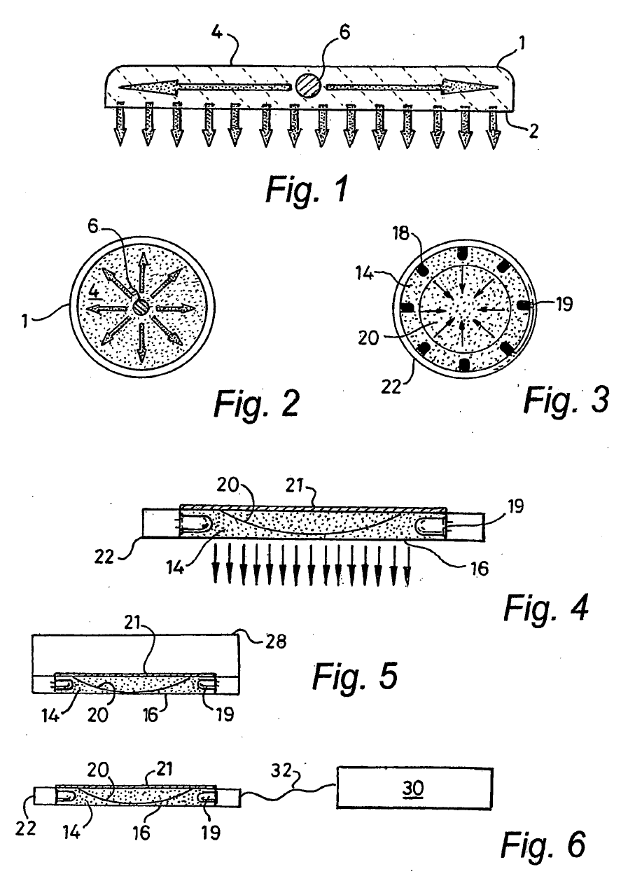

The ambulatory device of FIG. 1 is intended for use in providing electromagnetic radiation to an area of the skin of a patient, as part of a therapeutic and/or cosmetic treatment.

The device comprises a diffusing member 1 which takes the form of a disc of a diffusing material. The diffusing material can be any suitable semi-transparent material, for example, a suitable plastics material. In this case, the light diffusing material is nylon 66, perspex, acetate or silicone.

The underside of the member 1 defines a flat circular output surface 2 through which the device emits light, and which, in use, covers the area to be treated. The upper surface 4 of the disc 1 may carry a reflective coating for reflecting light which would otherwise escape through the top of the disc 1 back down towards the output surface 2.

This embodiment of device has a single light source in the form of a centrally mounted light emitting diode (LED) 6. The LED 6 is wholly embedded in the centre of the disc 1, the disc including passages (not shown) to enable electrical connections to the LED to be made through the top of the disc 1. Although the LED 6 is shown as a circular component, it is of a conventional shape for an LED, i.e. a short cylindrical rod having a domed front surface and contacts for connection to an electrical power supply at the rear. The LED is vertically orientated within the disc 1 so that the domed surface faces downwards, the cylindrical wall of the LED is vertical and the surface on which the contacts are provided is uppermost.

In use, the LED 6 emits light directly towards the surface 2 and sideways. Since the LED is embedded within the disc 1, all of its light is acted on by the diffusing material. The diffusing material is preferably able to scatter the emitted light to an extent sufficient to achieve even distribution of light across the output surface 2, but is not so great as to block light travelling within the disc 1.

Thus the light emitted by a single light source can provide even illumination over a surface of a much larger area than that of the source. Accordingly, a single light source can be used in the treatment of a relatively large area. The device is, in use, connected to a separate power supply and control unit for controlling the operation of the LED. The power supply and the control unit are not shown in FIGS. 1 and 2, but because they only power a single LED they can be of a relatively lightweight and compact construction and could be easily attached to the patient or to the device itself.

The power source and control means can take the form of batteries connected to control electronics incorporating a control for time of exposure, including the possibility of a delayed start to allow a photopharmaceutical to be metabolised into its photoactive form. Controls for brightness and pulsing may also be included. The device could generate an irradiance in the range 0-10 mW/cm², which is considerably lower than those generated by conventional sources such as lasers and filtered lamps, as these typically generate irradiances in the region 75-150 mWcm².

The device can be supplied with means for attaching it to a patient. One example of such a means would be transparent adhesive tape which extends over the surface 2 and beyond to provide adhesive surfaces for attaching the device to a patient. Prior to attachment, these surfaces could be protected by removal plastics films.

The device could be used for a range of pre-malignant, malignant and inflammatory diseases. Examples of pre-malignant skin disease are Bowen's Disease, Solar Keratosis, Arsenical Keratosis, Paget's Disease and Radiodermatitis. Malignant diseases include all types of Basal cell carcinomas, Squamous cell carcinomas, secondary metastases and cutaneous T-cell lymphomas. Inflammatory skin diseases include all types of dermatitis and psoriasis. Further diseases that are potential targets are a range of pre-malignant, malignant and non-cutaneous disorders such as primary and metastatic tumours, as well as inflammatory disorders, e.g. connective tissue disease, all type of arthritis, inflammatory bowel disease. The device can also be used in cosmetic treatments, for example the treatment of acne or anti-ageing and anti-wrinkle treatments.

A modified version of the device has a facility automatically to switch the source on and off so delivering the desired dose of radiation as a series of pulses. This can limit photobleaching and enables fresh uptake/metabolism of the photopharmaceutical within remaining viable target cells.

It will be appreciated that various modifications to the device may be made within the scope of the invention. Thus, for example, the diffusing material could be flexible so that the whole of the disc 1 can conform to the area to be treated. An example of such a diffusing material is partially vulcanised silicone. Furthermore, the LED 6 may be of a different shape or, instead of an LED 6, the device could have another type embedded light source such as a distributed element LED, a miniature fluorescent lamp or a miniature incandescent light bulb.

FIGS. 3-6 show an embodiment of device which is for use in similar situations to the embodiment shown in FIGS. 1 and 2, but which employs a plurality of light sources and has a diffusing member which is so shaped as to distribute more evenly the light emitted by the sources.

This embodiment of device has a disc-shaped diffusing member 14 having a flat circular underside 16 that acts as the output surface. Eight radial recesses are formed in the periphery of the disc 14, each recess extending from the edge of the disc towards its centre, and each accommodating a respective one of eight LEDs, for example, LEDs 18 and 19, each of which is identical to LED 18, and which are equiangularly arranged around the periphery of the disc 14. The upper surface 20 of the disc 14 has a central concave portion so that the thickness of the disc progressively decreases from its periphery to its centre. An annular plastics c-sectioned housing 22 extends around the periphery of the disc 14. As can be seen in FIG. 9, the housing and the disc define an annular cavity 24 which accommodates the electrical connections for the LEDs (such as the connection 26 for the LED 18). As is shown in FIG. 3, the LEDs are connected in series. The housing includes contacts (not shown) connecting the LEDs to a power supply, two examples of which are shown in FIGS. 5 and 6. In FIG. 5, the power supply takes the form of a disc-shaped housing 28 mounted directly on top of the disc 14 and housing 22. The housing 28 contains batteries for the power supply and a control box providing similar control to the controller described in relation to the first embodiment. In the arrangement shown in the FIG. 6, the housing for the batteries and control electronics is referenced 30 and is separate from the disc 14 and housing 22. In this case, the batteries and control electronics are connected to the device via a cord 32.

The disc 14 may be of any of the materials used for the disc 1 in the first embodiment. The upper surface of the disc carries a reflective layer 21 for reflecting light that would otherwise escape through the top of the disc back into the diffusing material.

The LEDs are arranged radially relative to the disc 14, and emit light that is directed sideways and towards the centre of the disc as indicated by the solid, radial arrows in FIG. 3. The disc diffuses the emitted light, some of which is reflected from the reflective layer 21, causing the light to be emitted from the underside of the disk, as indicated by the vertical arrows of FIG. 4. The concave portion of the upper surface 20 of the disc compensates for the drop in intensity of light with distance from the LEDs so that the whole of the output surface 16 is substantially evenly illuminated by the LEDs.

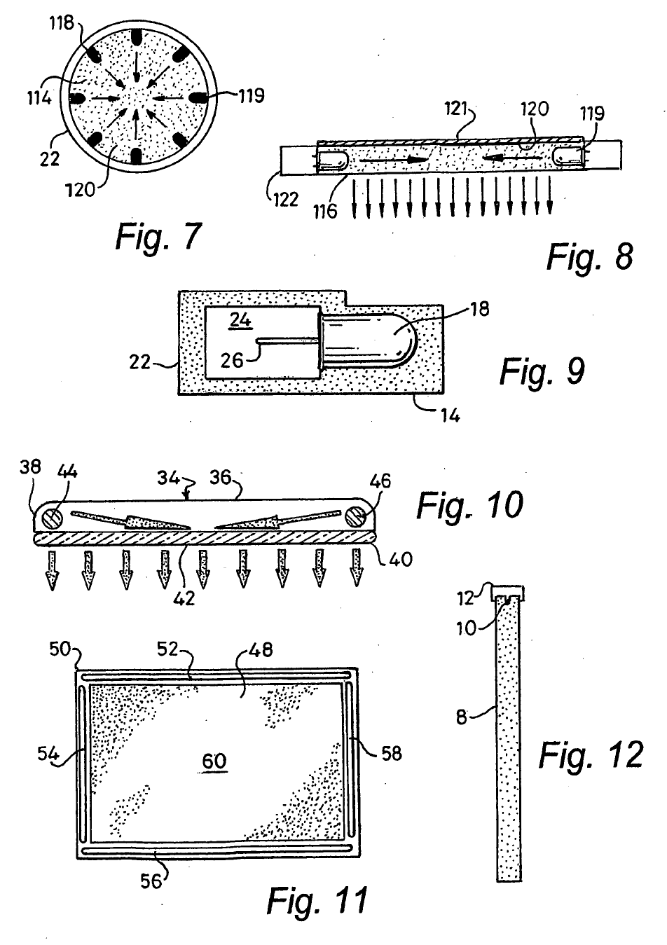

The embodiment shown in FIGS. 7 and 8 is very similar to that shown in FIGS. 3 to 6, and the corresponding components have therefore been denoted by the reference numerals of FIGS. 3 to 6 raised by 100. Thus, in this case, eight LEDs, each identical to LED 6, are radially arranged around the periphery of a diffusing disc 114 which is encircled by an annular housing 122 and which has an upper circular reflective layer 121. However, this embodiment differs from that shown in FIGS. 5-6 in that the disc 114 is a planar, the upper and lower circular faces 116 and 121 being parallel with each other.

If a current of 160 mA is supplied to the device of FIG. 7 at a voltage of 7.5 volts, the device emits light of a brightness of 3000 cd/m² at the centre of the diffusing disc 114.

The design of the embodiments shown in FIGS. 3-6, 7 and 8 may be varied with the device having a selected number of LEDs between one and eight. Each configuration of LEDs has a respective arrangement of electrical connections connecting the LEDs in series. Where power requirements are an issue, and homogeneity of illumination is not as important, the arrangement of source(s) may be asymmetric (eg, just one LED). The same designed diffusing member may be used for all the possible numbers of LEDs since one or more recesses can be vacant where fewer than eight LEDs are to be used, LEDs only being placed in selected recesses.

The embodiment of device shown in FIG. 10 differs from the previously described embodiments in that, instead of being embedded in the diffusing member, a number of LEDs are situated above the diffusing member so as to shine on to the latter.

The device comprises a housing 34 of Nylon 66, Silicone or PET (Polyethylene Terephthalate) having a circular top 36 from which a cylindrical apron 38 depends. Attached to the bottom of the apron 38 is a diffusing member in the form of a disc 40 which may be of any of the materials constituting the diffusing members of the other embodiments described above. The lower surface of the disc 40, reference 42, constitutes a circular output surface for lighting by the device.

The housing 34 and disc 40 define a cavity 42 which contains eight LEDs, two of which are shown at 44 and 46, equi-angularly arranged around the periphery of the cavity 42. As is indicated in FIG. 10, the light from the LEDs shines onto the diffusing disc 40 which distributes that light substantially evenly across the surface 42. The interior of the housing 34 has a reflective coating to maximise the amount of light shone onto the diffuser.

As before, the design may be varied so that the device has a selected number of LEDs between one and eight.

In the arrangement shown in FIG. 11, the diffusing member takes the form of a plate 48 of diffusing material (of any of the types of diffusing material constituting the other diffusing members described herein). This is surrounded by a rectangular housing 50 formed from a series of c-sectioned plastics extrusions which accommodate four cold cathode fluorescent lights 52, 54, 56 and 58. The fluorescent lights are directly aligned with the edges of the plate 48 so that the light that they emit passes directly into the plate which distributes that light over an output surface constituted by the rectangular face 60 of the plate.

The lamps are connected to a power supply and control unit which is similar to the power supplies and control units described in relation to the other embodiments, but which does include an inverter for converting DC power from the batteries to AC for operating the lamps.

The device shown in FIG. 12 is a light emitting rod or cylinder for use inside the esophagus or another internal body structure. In this case, the diffusing member takes the form of a cylindrical rod 8 of a radius of 1.25-2.25 cm and a length of 10-12 cm (it will be appreciated that different dimensions may be selected depending on the intended use of the device). One end face of the rod 8 is recessed so as to accommodate a light source in the form of an LED 10 embedded therein. The same face is covered by an electrical housing 12 which provides external terminals (not shown) through which the LED 10 is connected to an external power-source (not shown). The housing takes the form of a cylindrical plastics cup which is spaced from the top of the rod 8 so as to define a cavity that accommodates electrical wiring connecting the contacts on the LED 10 to the terminals for connection to the power supply. The rod may be formed from any of the diffusing materials used to constitute the disc 1 of the embodiment shown in FIGS. 1 and 2. Alternatively, the rod could be formed from a material which is flexible.

The LED is orientated with its domed-front end lowermost and its contacts at the top of the device, so that the LED emits light downwards and sidewards into the rod 8. This light is diffused to provide substantially continuous, preferably even, illumination along the length of the rod. More than one LED may be provided to ensure that the required lightness is achieved.

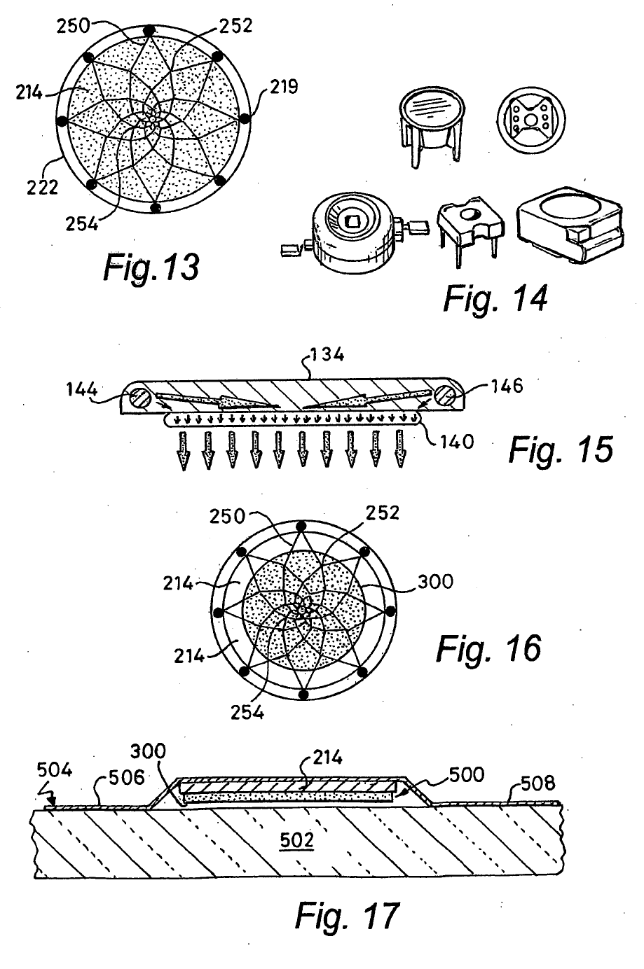

The device shown in FIG. 13 is similar in many respects to that shown in FIGS. 3 to 6, and the corresponding components have therefore been denoted by the reference numerals of FIGS. 3 to 6, raised by 200. Thus this embodiment has the same arrangement of peripheral radial LEDs as are used in the FIG. 3-6 embodiment, connected together in the same way as those of the FIGS. 3-6 embodiment. The device also has a reflective top layer (not shown), but this forms part of a disc 214 of light guide material of the type shown in FIG. 5 of WO2005/01070.

The disc has a network of light guides, e.g. 250, pairs of which extend from each respective light source towards the centre of the disc. The light guides cross at numerous crossing points, e.g. 252 and 254, distributed across the disc 214. Light ‘leaks’ out from the guides at these points to provide distributed illumination of the disc. The number density of these points increases towards the disc centre to compensate for the inverse relationship between the intensity of the light being conveyed by the light guides with distance from the sources.

The types of light source used by the various embodiments of invention described above are only examples, it being within the scope of the invention to use different types of light source. In particular, where the or each light source comprises an LED, it does not have to be of the shape (domed cylinder) of the LEDs used in all but the fifth embodiment. FIG. 14 shows examples of other shapes of LED which may be used. Where these are embedded in the diffusing member each is accommodated in an appropriately shaped cavity in the diffusing member.

Although the embodiment in FIG. 1 is intended to generate an irradiance of 0-10 W/cm², it is believed to be possible to drive any of the described embodiments in such a way that they produce irradiances up to 75 W/cm².

The embodiments which have multiple LEDs may be modified by the provision of LEDs of different colours arranged around the diffusing member in a repeating (for example, alternating where the LEDs are of two different colours) sequence. This allows treatment at different depths. To that end the control and power connections of the LEDs of these versions are such that different colour LEDs may be activated and deactivated at different times.

FIG. 15 shows a modified version of the device shown in FIG. 10 and uses the reference numerals of FIG. 10, raised by 100 to denote corresponding components.

Instead of being contained in a hollow housing, the LEDs are embedded within a solid diffusing member 134, formed from any of the diffusing substances in which the LEDs are embedded in the embodiment described above. The exterior shape of the diffusing member 134 is the same as that of the housing of the FIG. 10 embodiment, the underside of the diffusing member 134 thus being circular. The diffusing disc 140 is attached to the bottom of the diffusing member 134, but in this case is of a slightly smaller diameter than the underside of the member 134.

In use, light from the LEDs is distributed across the width of the disc 140 by the first diffusing member 134. The disc 140 further distributes that light across the area of the diffusing member to facilitate homogenous light extraction across an output surface constituted by the underside of the disc 140. The outer surface of the disc 140 is relatively rough, so as to assist in the extraction process.

The disc 140 may be attached to the underside of the member 134 by any suitable means, for example a transparent adhesive.

A similar double diffusion and light extraction structure is employed in the embodiment shown in FIG. 16.

FIG. 16 shows the FIG. 13 embodiment, in which a second diffuser, in the form of a disc 300, is attached to the underside of the disc 214 of light guide material (for example by means of an adhesive which is transparent to the wavelength of emitted light), so as to be in intimate contact with the disc. The disc 300 is substantially identical to the disc 140 shown in FIG. 15.

In order to facilitate ambulatory use of the devices for use in treating the skin of a patent, those devices may be provided with attachment means, an example of which is shown in FIG. 17.

In FIG. 17, the reference numeral 500 generally denotes a device having light sources and a diffusing member through which light passes from the sources to the skin 502 of a patent. This device, in the present example, is the device shown in FIG. 16, although any of the first to fourth and sixth embodiments could be used with the attachment means shown in FIG. 17. The attachment means comprises a piece of single sided adhesive tape 504 which extends over the back of the diffusing member 214 to adhere the tape to the diffusing member. The tape 503 also extends beyond the diffusing member to provide side portions 506 and 508 which are pressed against the skin 502 to adhere the tape, and hence the device, in position on the skin 502. Optionally, the underside of the disc 300 (i.e. the output surface) carries an adhesive layer which also serves to stick the device to the skin, thus facilitating attachment of the device. This layer is of an adhesive which is substantially transparent to the wavelength of radiation emitted by the device to cause the treatment of the skin.

1. An ambulatory device for use in therapeutic and/or cosmetic treatment, the device comprising a localized light source and a diffusing member for distributing light from the source over an area to be treated so as to illuminate, and cause said treatment of, that area.

2. The device according to claim 1, in which the diffusing member is made from a flexible material so that it is capable of conforming to the area to be treated.

3. The device according to claim 1, in which the diffusing member has an output surface which, in use, covers the area to be treated, said surface defining an emitting area across which light from the source is emitted by the device.

4. The device according to claim 3, in which the source is situated behind said output surface so that substantially all the light emitted by the device passes through at least part of the diffusing member, the area of the output surface being greater than that of the source.

5. The device according to claim 3, in which the output surface has an area of at least one square centimetre.

6. The device according to claim 5, in which the size of said area is in the range of 3-400 cm².

7. The device according to claim 1, in which the light source is at least partially accommodated in a recess in the diffusing member.

8. The device according to claim 7, in which the light source is preferably embedded within the diffusing member.

9. The device according to claim 3, in which the device has a single light source, symmetrically situated relative to the output surface.

10. The device according to claim 3, in which the light source is one of a plurality of such light sources.

11. The device according to claim 10, in which the light sources are arranged in an array which is symmetrical relative to the output surface.

12. The device according to claim 10, in which each source is at least partially accommodated in a respective recess in the diffusing member.

13. The device according to claim 12, in which each source is embedded in the diffusing member.

14. The device according to claim 10, in which the possible number of sources in the device lies in the range of 1 to 12 inclusive.

15. The device according to claim 1, in which the or each source is situated at the periphery of the diffusing member.

16. The device according to claim 1, in which the or each source comprises a light emitting semi-conductor.

17. The device according to claim 16, in which the or each source comprises an inorganic light emitting diode.

18. The device according to claim 1, in which the diffusing member comprises a substantially homogenous body of translucent material all of which will scatter the light.

19. The device according to claim 1, in which the diffusing member has one or more light guides for supplying light to emission zones distributed across the member.

20. The device according to claim 1, in which the diffusing member comprises a sheet of diffusing material, having a flat face.

21. The device according to claim 20, in which the thickness of the sheet decreases with increasing distance from the source or sources so as to compensate for the inverse relationship between the intensity of the light emitted from the source or sources with distance therefrom.

22. The device according to claim 21, in which said decrease in thickness is progressive.

23. The device according to claim 22, in which the device has a plurality of sources arranged around the periphery of the diffusing member, the latter having a concave surface.

24. The device according to claim 1, in which the diffusing member comprises a rod, the light source being situated at one end of the rod.

25. The device according to claim 24, in which the rod is flexible.

26. The device according to claim 10, in which the light sources are operable to emit light

27. The device according to claim 1, in which the device includes attachment means for attaching the device to a user.

28. The device according to claim 27, in which said attachment means comprises an adhesive surface and/or bandage.

29. The device according to claim 1, further comprising a photopharmaceutical preparation.

30. The device according to claim 29 wherein the photopharmaceutical preparation comprises an inactive compound which is metabolised in vivo to an active compound.
